(19) United States
(12) Patent Application Publication
(10) Pub. No.: US 2024/0096498 A1
(43) Pub. Date: Mar. 21, 2024

(54) METHOD AND SYSTEM FOR EVALUATING RISK OF SUBJECT GETTING SPECIFIC DISEASE

(71) Applicant: GRAPHEN, INC., New York, NY (US)

(72) Inventors: Yi-Ting CHEN, Taipei City (TW); Sing-Han HUANG, Taipei City (TW); Ching-Yung LIN, Scarsdale, NY (US); Xiang-Yu LIN, Taipei City (TW); Cheng-Tang WANG, Taipei City (TW); Raksha NANDANAHOSUR RAMESH, New York, NY (US); Pei-Hsin CHEN, New York, NY (US)

(21) Appl. No.: 18/457,206

(22) Filed: Aug. 28, 2023

Related U.S. Application Data
(60) Provisional application No. 63/407,120, filed on Sep. 15, 2022.

(51) Int. Cl. G16H 50/30; G16B 20/20; G16H 50/70; G16H 70/60

(52) U.S. Cl. CPC G16H 50/30; G16B 20/20; G16H 50/70; G16H 70/60; G16H 50/80

(57) ABSTRACT

A method for evaluating a risk of a subject getting a specific disease includes steps of: storing a reference database that contains original parameter sets; selecting target alleles from an SNP profile derived from genome sequencing data of a subject; selecting target parameter sets from among the original parameter sets; calculating, for each of the target parameter sets, a race factor based on a global risk allele frequency and a group-specific risk allele frequency included in the target parameter set; calculating a genetic factor based on statistics, global reference allele frequencies, the race factors for the target parameter sets, and numbers of chromosomes in homologous chromosome pairs included in the target parameter sets; calculating a citation factor based on numbers of citation times included in the target parameter sets; and calculating a risk score based on the genetic factor and the citation factor.

### CROSS-REFERENCE TO RELATED APPLICATION

This application claims the benefit of U.S. Provisional Patent Application No. 63/407,120, filed on Sep. 15, 2022, which is incorporated by reference herein in its entirety.

### FIELD

The disclosure relates to a method and a system for evaluating a risk of a subject getting a specific disease.

### BACKGROUND

Genes related to hereditary genetic disorders (e.g., cystic fibrosis, haemophilia and congenital heart disease) and non-hereditary genetic disorders (e.g., skin cancers, lung carcinoma and colorectal cancer caused by environmental factors) may be identified using genome sequencing.

### SUMMARY

Therefore, an object of the disclosure is to provide a method and a system for scoring a risk of a subject getting a specific disease.

According to one aspect of the disclosure, the method includes steps of: establishing a reference database by collecting data from a medical literature database, an allele frequency database, and a plurality of databases that compiles data of genome-wide association study (GWAS), the reference database containing M number of original parameter sets that respectively correspond to M number of specific risk alleles respectively at M number of chromosomal positions where single-nucleotide polymorphisms (SNPs) related to the specific disease occur, M being a positive integer greater than one, each of the M number of original parameter sets including a plurality of statistics related to the corresponding one of the M number of specific risk alleles, a global risk allele frequency that is related to an allele frequency of the corresponding one of the M number of specific risk alleles in global population, a group-specific risk allele frequency that is related to an allele frequency of the corresponding one of the M number of specific risk alleles in a certain race group, a global reference allele frequency that is related to the global risk allele frequency, a number of citation times that literatures related to the corresponding one of the M number of specific risk alleles are cited, and a number of chromosomes in a homologous chromosome pair having the corresponding one of the M number of specific risk alleles; selecting, from an SNP profile derived from genome sequencing data of the subject, N number of target alleles that respectively match N number of specific risk alleles in the M number of specific risk alleles included in the reference database, N being a positive integer not greater than M; selecting, from among the M number of original parameter sets, N number of target parameter sets that correspond respectively to the N number of specific risk alleles; for each of the N number of target parameter sets, calculating a race factor based on the global risk allele frequency and the group-specific risk allele frequency of the target parameter set; calculating a genetic factor based on the statistics respectively of the N number of target parameter sets, the global reference allele frequencies respectively of the N number of target parameter sets, the race factors respectively calculated for the N number of target parameter sets, and the numbers of chromosomes in homologous chromosome pairs of the N number of target parameter sets; calculating a citation factor based on the numbers of citation times respectively of the N number of target parameter sets; and calculating a risk score based on the genetic factor and the citation factor.

According to another aspect of the disclosure, the system includes a storage, a receiving module, and a processor that is electrically connected to the storage and the receiving module.

The storage is configured to store a reference database that is established in advance by collecting data from a medical literature database, an allele frequency database, and a plurality of databases that compiles data of GWAS. The reference database contains M number of original parameter sets that respectively correspond to M number of specific risk alleles respectively at M number of chromosomal positions where SNPs related to the specific disease occur, where M is a positive integer greater than one. Each of the M number of original parameter sets includes a plurality of statistics related to the corresponding one of the M number of specific risk alleles, a global risk allele frequency that is related to an allele frequency of the corresponding one of the M number of specific risk alleles in global population, a group-specific risk allele frequency that is related to an allele frequency of the corresponding one of the M number of specific risk alleles in a certain race group, a global reference allele frequency that is related to the global risk allele frequency, a number of citation times that literatures related to the corresponding one of the M number of specific risk alleles are cited, and a number of chromosomes in a homologous chromosome pair having the corresponding one of the M number of specific risk alleles.

The receiving module is configured to receive an SNP profile derived from genome sequencing data of the subject.

The processor is configured to implement a method that includes steps of: selecting, from the SNP profile derived from genome sequencing data of the subject, N number of target alleles that respectively match N number of specific risk alleles in the M number of specific risk alleles indicated in the reference database, N being a positive integer not greater than M; selecting, from among the M number of original parameter sets, N number of target parameter sets that correspond respectively to the N number of specific risk alleles; for each of the N number of target parameter sets, calculating a race factor based on the global risk allele frequency and the group-specific risk allele frequency of the target parameter set; calculating a genetic factor based on the statistics respectively of the N number of target parameter sets, the global reference allele frequencies respectively of the N number of target parameter sets, the race factors respectively calculated for the N number of target parameter sets, and the numbers of chromosomes in homologous chromosome pairs of the N number of target parameter sets; calculating a citation factor based on the numbers of citation times respectively of the N number of target parameter sets; and calculating a risk score based on the genetic factor and the citation factor.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a system for evaluating a risk of a subject getting a specific disease according to an embodiment of the disclosure.

FIG. 2 is a schematic view illustrating original parameter sets contained in a reference database of the system according to the embodiment of the disclosure.

FIG. 3 is a flow chart illustrating a method for evaluating a risk of a subject getting a specific disease according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

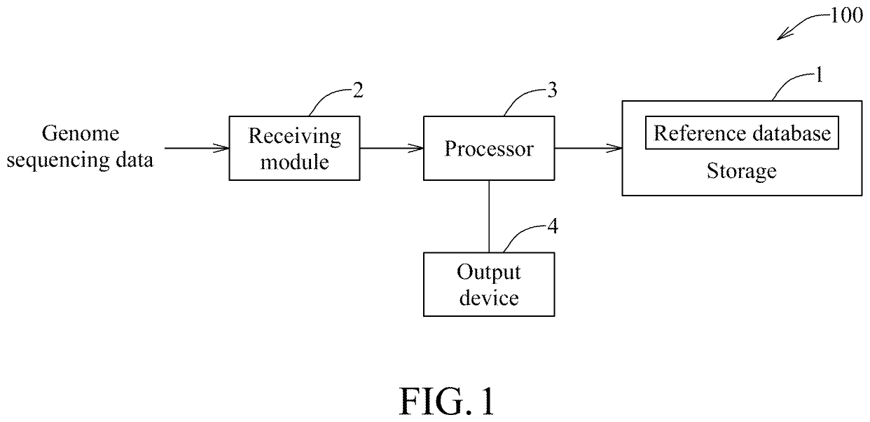

Referring to FIG. 1, an embodiment of a system 100 for evaluating a risk of a subject getting a specific disease according to the disclosure is illustrated. The system 100 may be implemented by a desktop computer, a laptop computer, a notebook computer or a tablet computer, but implementation thereof is not limited to what are disclosed herein and may vary in other embodiments. The system 100 includes a storage 1, a receiving module 2, and a processor 3 that is electrically connected to the storage 1 and the receiving module 2.

The storage 1 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto. The storage 1 is configured to store a reference database that is established in advance by collecting data from a medical literature database, an allele frequency database, and a plurality of databases that compiles data of genome-wide association study (GWAS). The databases that compiles data of GWAS exemplarily include the GWAS Catalog (www.ebi.ac.uk/gwas), the single nucleotide polymorphism database (dbSNP, https://www.ncbi.nlm.nih.gov/snp/) and the ClinVar (https://www.ncbi.nlm.nih.gov/clinvar/). The databases that compiles data of GWAS collect, from resources of academic publication and clinical research, data that are related to association between single-nucleotide polymorphism (SNP)/single-nucleotide variant (SNV) and disease (including pathogenicity, clinical severity and symptoms). The allele frequency database is exemplarily the Allele Frequency Aggregator (ALFA, https://www.ncbi.nlm.nih.gov/snp/docs/gsr/alfa/). The allele frequency database collects data that are related to allele frequencies of alleles from 12 diverse populations in different regions around the world, facilitating studies conducted on impact of variations of alleles on variations of genotypes and phenotypes with respect to regional differences and/or racial disparities. The medical literature database is exemplarily the MEDLINE database that can be accessed by using the PubMed® search engine (https://pubmed.ncbi.nlm.nih.gov/). It is worth to note that in the GWAS Catalog, the dbSNP and the ClinVar, a reference SNP (rs) number (also known as an SNP identifier, SNP ID) having a format of letters “rs” followed by a number is used as a keyword to search relevant information about a specific SNP (e.g., a chromosome number where the specific SNP occurs), a locus where the specific SNP occurs, nucleotide types involved in the specific SNP for a reference human genome derived from Americans, nucleotide types of a risk allele involved in the specific SNP, and a gene name of a gene involved in the specific SNP.

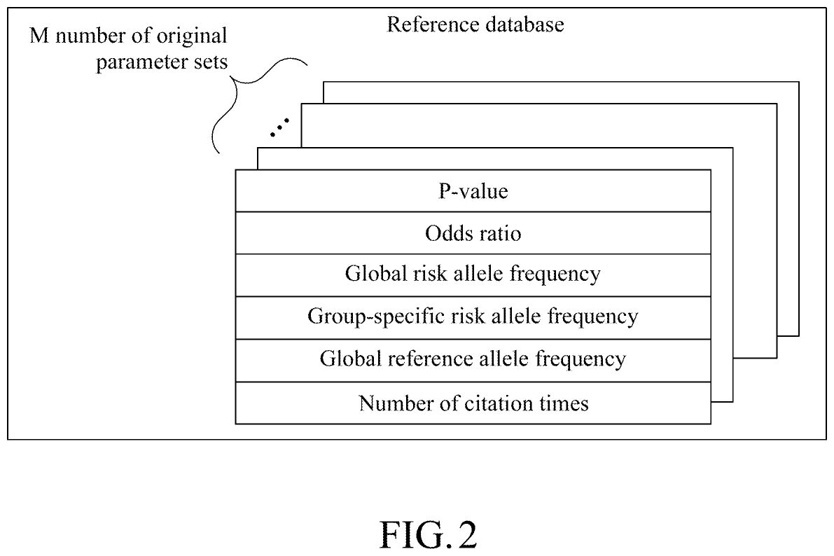

Referring to FIG. 2, the reference database contains M number of original parameter sets that respectively correspond to M number of specific risk alleles respectively at M number of chromosomal positions where SNPs related to the specific disease occur, wherein M is a positive integer greater than one. Each of the M number of original parameter sets includes a plurality of statistics related to the corresponding one of the M number of specific risk alleles, a global risk allele frequency that is related to an allele frequency of the corresponding one of the M number of specific risk alleles in global population, a group-specific risk allele frequency that is related to an allele frequency of the corresponding one of the M number of specific risk alleles in a certain race group, a global reference allele frequency that is related to the global risk allele frequency, a number of citation times that literatures related to the corresponding one of the M number of specific risk alleles are cited in the medical literature database, and a number of chromosomes in a homologous chromosome pair having the corresponding one of the M number of specific risk alleles. In particular, for each of the M number of original parameter sets, the statistics include a p-value and an odds ratio. The p-value represents a probability that an association between the specific disease and the corresponding one of the M number of specific risk alleles is due to random chance. The less the p-value, the less the probability that an association between the specific disease and the corresponding one of the M number of specific risk alleles is due to random chance. The odds ratio is a ratio of a probability of a person with the corresponding one of the M number of specific risk alleles getting the specific disease to a probability of a person without the corresponding one of the M number of specific risk alleles getting the specific disease. The greater the odds ratio, the higher the probability of a person with the corresponding one of the M number of specific risk alleles getting the specific disease. It is worth to note that a sum of the global risk allele frequency and the global reference allele frequency is equal to one.

In a scenario where the specific disease is esophageal carcinoma, for database versions of October 2020, the GWAS Catalog, the dbSNP and the ClinVar have collected relevant data for 302 SNPs that respectively correspond to 302 SNP IDs. By summarizing the aforesaid relevant data for the 302 SNPs obtained from the GWAS Catalog, the dbSNP and the ClinVar based on conditions and results of relevant experiments related to the SNPs and publications of the relevant experiments, and by incorporating data that are obtained from the ALFA (having a database version of October 2020) and that are related to allele frequencies of alleles involved in the 302 SNPs, the reference database is established to contain 14 (i.e., M=14) original parameter sets that respectively correspond to 14 specific risk alleles which are related to esophageal carcinoma and which are respectively involved in 14 SNPs respectively corresponding to 14 SNP IDs.

In some embodiments, the receiving module 2 may be, but not limited to, a network interface controller or a wireless transceiver that supports wireless communication standards, such as Bluetooth® technology standards, Wi-Fi technology standards and/or cellular network technology standards, and is configured to receive an SNP profile that is derived from genome sequencing data of the subject and that is transmitted by a remote electronic device (e.g., a computer). In other embodiments, the receiving module 2 is a physical connector (e.g., a USB connector), and is configured to receive the SNP profile from an external electronic device (e.g., a flash drive) that is electrically connected to the receiving module 2.

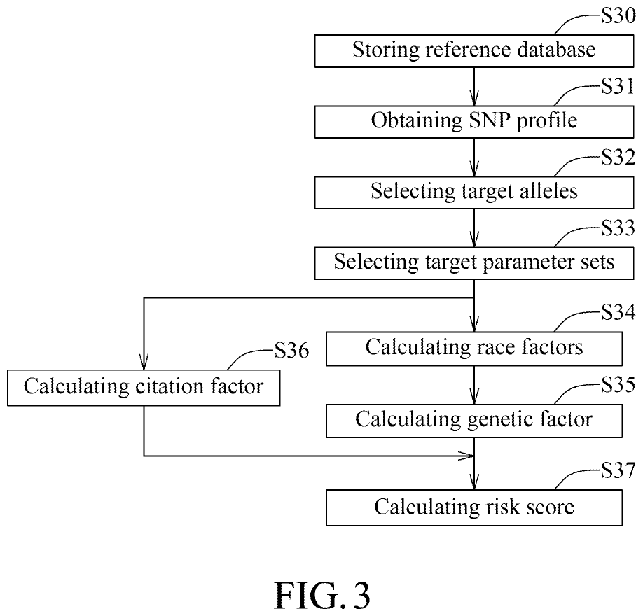

The processor 3 may be implemented by a central processing unit (CPU), a microprocessor, a micro control unit (MCU), a system on a chip (SoC), or any circuit configurable/programmable in a software manner and/or hardware manner so as to implement functionalities discussed in this disclosure. The system 100 is configured to implement a method for evaluating a risk of a subject getting a specific disease according to the disclosure. Referring to FIG. 3, the method includes steps S30 to S37 delineated below.

In step S30, the system 100 stores the reference database in the storage 1.

In step S31, the processor 3 obtains, from the receiving module 2, the SNP profile derived from genome sequencing data of the subject.

In step S32, the processor 3 selects, from the SNP profile derived from genome sequencing data of the subject, N number of target alleles that respectively match N number of specific risk alleles in the M number of specific risk alleles indicated in the reference database, where N is a positive integer not greater than M. For example, comparing the SNP profile with the 14 original parameter sets described previously, 7 target alleles (i.e., N=7) are selected and shown in Table 1 below.

TABLE 1

| Variant | Chromosome | Chromosome | | Risk | |
|---|---|---|---|---|---|
| No. | number | start | Allele | allele | SNP ID |
| 1 | 12 | 112241766 | GA | A | rs671 |
| 2 | 21 | 36357861 | GG | G | rs2014300 |
| 3 | 12 | 112168009 | TA | A | rs11066015 |
| 4 | 10 | 96058298 | CT | T | rs37665524 |
| 5 | 10 | 96066341 | AG | G | rs2274223 |
| 6 | 12 | 112817783 | TA | A | rs11066280 |
| 7 | 5 | 148904092 | TT | T | rs100588728 |

It is worth to note that for each of the 7 target alleles, a number of chromosomes in a homologous chromosome pair having the corresponding one of the 7 target alleles can be inferred from Table 1. Specifically, numbers of chromosomes in homologous chromosome pairs for the 7 target alleles (respectively in variant Nos. 1 to 7 in Table 1) are one, two, one, one, one, one and two, respectively.

In step S33, the processor 3 selects, from among the M number of original parameter sets, N number of target parameter sets that correspond respectively to the N number of specific risk alleles. For example, 7 target parameter sets are selected from the 14 original parameter sets described previously, and are shown in Table 2 below. It should be noted that in Table 2a and Table 2b, −log₁₀ P represents a logarithm of a reciprocal of the p-value with respect to base 10, and the group risk allele frequency is for the population in East Asia.

TABLE 2a

| Data | Chromosome | Chromosome | | | Odds |
|---|---|---|---|---|---|
| No. | number | start | SNP ID | −log₁₀P | ratio |
| 1 | 12 | 112241766 | rs671 | 23.523 | 1.67 |
| 2 | 21 | 36357861 | rs2014300 | 21.097 | 1.43 |
| 3 | 12 | 112168009 | rs11066015 | 20.155 | 1.38 |
| 4 | 10 | 96058298 | rs37665524 | 8.699 | 1.35 |
| 5 | 10 | 96066341 | rs2274223 | 19.398 | 1.34 |
| 6 | 12 | 112817783 | rs11066280 | 14.699 | 1.30 |
| 7 | 5 | 148904092 | rs100588728 | 8.301 | 2.04 |

TABLE 2b

| | Global | Global | | |
|---|---|---|---|---|
| | risk | reference | Group | |
| Data | allele | allele | risk allele | Citation |
| No. | frequency | frequency | frequency | times |
| 1 | 0.006 | 0.994 | 0.218 | 160 |
| 2 | 0.849 | 0.151 | 0.900 | 175 |
| 3 | 0.010 | 0.990 | 0.213 | 175 |
| 4 | 0.288 | 0.712 | 0.202 | 298 |
| 5 | 0.322 | 0.678 | 0.217 | 175 |
| 6 | 0.008 | 0.992 | 0.224 | 175 |
| 7 | 0.537 | 0.463 | 1.000 | 175 |

In step S34, for each of the N number of target parameter sets, the processor 3 calculates a race factor based on the global risk allele frequency and the group-specific risk allele frequency of the target parameter set. Specifically, for an i^{th} one of the target parameter sets that corresponds to an i^{th} one of the N number of specific risk alleles, where i is an integer ranging from one to N, the processor 3 calculates the race factor according to a first formula and a second formula:

$${Factor}_{R a c e , i} = \{ \begin{matrix}{log}_{10} {Frequency\_ratio}_{Group risk , i} + 1 , {log}_{10} {Frequency\_ratio}_{Group risk , i} \geq 0 \\ \frac{1}{1 - {log}_{10} {Frequency\_ratio}_{Group risk , i}} , {log}_{10} {Frequency\_ratio}_{Group risk , i} < 0\end{matrix} ;$$

$$and {Frequency\_ratio}_{Group risk , i} = \frac{{Frequency}_{Group risk , i}}{{Frequency}_{G l obal risk , i}} ,$$

where Factor_{Race,i} represents the race factor for the i^{th} one of the N number of specific risk alleles, Frequency_{Group risk,i} represents the group-specific risk allele frequency for the i^{th} one of the N number of specific risk alleles, and Frequency_{Global risk,i} represents the global risk allele frequency for the i^{th} one of the N number of specific risk alleles. For example, based on the data in Tables 2a and 2b, the processor 3 calculates Frequency_ratio_{Group risk,1} to Frequency_ratio_{Group risk,7} as shown in Table 3 below, and calculates Factor_{Race,1} to Factor_{Race,7} as shown in Table 4 below.

TABLE 3

| | |
|---|---|
| Frequency_ratio_{Group risk, 1} | 36.317 |
| Frequency_ratio_{Group risk, 2} | 1.060 |
| Frequency_ratio_{Group risk, 3} | 21.735 |
| Frequency_ratio_{Group risk, 4} | 0.703 |
| Frequency_ratio_{Group risk, 5} | 0.672 |
| Frequency_ratio_{Group risk, 6} | 27.329 |
| Frequency_ratio_{Group risk, 7} | 1.864 |

TABLE 4

| | |
|---|---|
| Factor_{Race, 1} | 2.493 |
| Factor_{Race, 2} | 1.025 |
| Factor_{Race, 3} | 2.295 |
| Factor_{Race, 4} | 0.866 |
| Factor_{Race, 5} | 0.852 |
| Factor_{Race, 6} | 2.387 |
| Factor_{Race, 7} | 1.270 |

In step S35, the processor 3 calculates a genetic factor based on the statistics (i.e., groups of the p-values and the odds ratios) respectively of the N number of target parameter sets, the global reference allele frequencies respectively of the N number of target parameter sets, the race factors respectively calculated for the N number of target parameter sets, and the numbers of chromosomes in homologous chromosome pairs for the N number of specific risk alleles respectively of the N number of target parameter sets. Specifically, the processor 3 calculates the genetic factor according to a third formula:

$${Factor}_{Genetic} = \frac{1}{M} {Σ}_{i = 1}^{N} \frac{- {log}_{10} {P}_{i} \times {OR}_{i} \times {SNP\_Type}_{i} \times {Factor}_{R ace , i}}{{Frequency}_{G l obal ref , i}} ,$$

where Factor_{Genetic} represents the genetic factor, Pᵢ represents the p-value for an i^{th} one of the N number of specific risk alleles, ORᵢ represents the odds ratio for the i^{th} one of the N number of specific risk alleles, SNP_Typeᵢ represents the number of chromosomes in a homologous chromosome pair having the i^{th} one of the N number of specific risk alleles, Factor_{Race,i} represents the race factor for the i^{th} one of the N number of specific risk alleles, and Frequency_{Global ref,i} represents the global reference allele frequency for the i^{th} one of the N number of specific risk alleles. It should be noted that the numbers of chromosomes in homologous chromosome pairs for the N number of specific risk alleles are equal to the numbers of chromosomes in homologous chromosome pairs for the 7 target alleles, respectively. For example, by substituting relevant values in Tables 1, 2a, 2b and 4 into the third formula, the processor 3 calculates the genetic factor as 54.2.

In step S36, the processor 3 calculates a citation factor based on the numbers of citation times respectively of the N number of target parameter sets. Specifically, the processor 3 calculates the citation factor according to on a fourth formula:

Factor_{citation}=lnΣᵢ₌₁^{N}(Citation_numᵢ+1),

where Factor_{citation} represents the citation factor, and Citation_numᵢ represents the number of citation times for an one of the N number of specific risk alleles. For example, based on the values in column “Citation times” in Table 2b, the processor 3 calculates the citation factor as 7.20.

It should be noted that step S36 can be independently executed in parallel to the execution of steps S34 and S35.

In step S37, the processor 3 calculates a risk score based on the genetic factor and the citation factor. Specifically, the processor 3 calculates the risk score according to a fifth formula:

$${Score}_{risk} = \{ \begin{matrix}100 , {Factor}_{Genetic} \times {Factor}_{Citation} > 100 \\ {Factor}_{Genetic} \times {Factor}_{Citation} , 0 < {Factor}_{Genetic} \times {Factor}_{Citation} \leq 100\end{matrix} ,$$

where Scoreᵣᵢₛₖ represents the risk score, Factor_{Genetic} represents the genetic factor, and Factor_{citation} represents the citation factor. For example, in the above-mentioned case, when the genetic factor is equal to 54.2 and the citation factor is equal to 7.20, the processor 3 calculates the risk score as 100.

In some embodiments, the system 100 further includes an output device 4 (e.g., a display) electrically connected to the processor 3, and the processor 3 controls the output device 4 to present the risk score calculated in step S37. Further, the risk score can be provided to a user as an evaluation index for obtaining genetic information about susceptibility genes related to varieties of diseases and about any potential risk of developing cancer(s).

In some embodiments, the reference database stored in the storage 1 further contains a plurality of additional parameter sets that are related to a variety of additional diseases. In this way, by using the same SNP profile derived from genome sequencing data of the subject, the processor 3 is capable of implementing the method according to the disclosure, i.e., to calculate a plurality of risk scores respectively for the additional diseases, and informing the subject of conditions about his/her health according to the risk scores.

To sum up, in the system and the method according to the disclosure, the risk score is calculated for the target alleles that are included in the SNP profile derived from genome sequencing data of a subject and that respectively match the risk alleles indicated in the reference database, which is established by collecting data from the medical literature database, the allele frequency database, and the databases that compiles data of GWAS. Calculation of the risk score incorporates factors that are related to genetics, race and numbers of citation times. Therefore, the risk score thus calculated may facilitate assessment of a risk of the subject getting a specific disease.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for evaluating a risk of a subject getting a specific disease, comprising steps of:
storing a reference database by collecting data from a medical literature database, an allele frequency database, and a plurality of databases that compiles data of genome-wide association study (GWAS), the reference database containing M number of original parameter sets that respectively correspond to M number of specific risk alleles respectively at M number of chromosomal positions where single-nucleotide polymorphisms (SNPs) related to the specific disease occur, M being a positive integer greater than one, each of the M number of original parameter sets including a plurality of statistics related to the corresponding one of the M number of specific risk alleles, a global risk allele frequency that is related to an allele frequency of the corresponding one of the M number of specific risk alleles in global population, a group-specific risk allele frequency that is related to an allele frequency of the corresponding one of the M number of specific risk alleles in a certain race group, a global reference allele frequency that is related to the global risk allele frequency, a number of citation times that literatures related to the corresponding one of the M number of specific risk alleles are cited, and a number of chromosomes in a homologous chromosome pair having the corresponding one of the M number of specific risk alleles;
selecting, from an SNP profile derived from genome sequencing data of the subject, N number of target alleles that respectively match N number of specific risk alleles in the M number of specific risk alleles included in the reference database, N being a positive integer not greater than M;
selecting, from among the M number of original parameter sets, N number of target parameter sets that correspond respectively to the N number of specific risk alleles;
calculating, for each of the N number of target parameter sets, a race factor based on the global risk allele frequency and the group-specific risk allele frequency of the target parameter set;
calculating a genetic factor based on the statistics respectively of the N number of target parameter sets, the global reference allele frequencies respectively of the N number of target parameter sets, the race factors respectively calculated for the N number of target parameter sets, and the numbers of chromosomes in homologous chromosome pairs of the N number of target parameter sets;
calculating a citation factor based on the numbers of citation times respectively of the N number of target parameter sets; and
calculating a risk score based on the genetic factor and the citation factor.

2. The method as claimed in claim 1, wherein for each of the M number of original parameter sets, the statistics include:
a p-value representing a probability that an association of the specific disease with the corresponding one of the M number of specific risk alleles is due to random chance; and
an odds ratio, which is a ratio of a probability of a person with the corresponding one of the M number of specific risk alleles getting the specific disease to a probability of a person without the corresponding one of the M number of specific risk alleles getting the specific disease.

3. The method as claimed in claim 2, wherein the step of calculating a genetic factor is to calculate the genetic factor according to a formula:
$${Factor}_{Genetic} = \frac{1}{M} {Σ}_{i = 1}^{N} \frac{- {log}_{10} {P}_{i} \times {OR}_{i} \times {SNP\_Type}_{i} \times {Factor}_{R ace , i}}{{Frequency}_{G l obal ref , i}} ,$$
where Factor_{Genetic} represents the genetic factor, Pᵢ represents the p-value for an i^{th} one of the N number of specific risk alleles, ORᵢ represents the odds ratio for the i^{th} one of the N number of specific risk alleles, SNP_Typeᵢ represents the number of chromosomes in a homologous chromosome pair having the i^{th} one of the N number of specific risk alleles, Factor_{Race,i} represents the race factor for the i^{th} one of the N number of specific risk alleles, and Frequency_{Global ref,i} represents the global reference allele frequency for the i^{th} one of the N number of specific risk alleles.

4. The method as claimed in claim 1, wherein for an i^{th} one of the target parameter sets that corresponds to an i^{th} one of the N number of specific risk alleles, i being an integer ranging from one to N, the step of calculating a race factor is to calculate the race factor according to formulas:
$${Factor}_{R a c e , i} = \{ \begin{matrix}{log}_{10} {Frequency\_ratio}_{Group risk , i} + 1 , {log}_{10} {Frequency\_ratio}_{Group risk , i} \geq 0 \\ \frac{1}{1 - {log}_{10} {Frequency\_ratio}_{Group risk , i}} , {log}_{10} {Frequency\_ratio}_{Group risk , i} < 0\end{matrix} ;$$
$$and {Frequency\_ratio}_{Group risk , i} = \frac{{Frequency}_{Group risk , i}}{{Frequency}_{G l obal risk , i}} ,$$
where Factor_{Race,i} represents the race factor for the i^{th} one of the N number of specific risk alleles, Frequency_{Group risk,i} represents the group-specific risk allele frequency for the i^{th} one of the N number of specific risk alleles, and Frequency_{Global risk,i} represents the global risk allele frequency for the i^{th} one of the N number of specific risk alleles.

5. The method as claimed in claim 1, wherein the step of calculating a citation factor is to calculate the citation factor according to a formula:
Factor_{citation}=lnΣᵢ₌₁^{N}(Citation_numᵢ+1),
where Factor_{citation} represents the citation factor, and Citation_numᵢ represents the number of citation times for an i^{th} one of the N number of specific risk alleles.

6. The method as claimed in claim 1, wherein the step of calculating a risk score is to calculate the risk score according to a formula:
$${Score}_{risk} = \{ \begin{matrix}100 , {Factor}_{Genetic} \times {Factor}_{Citation} > 100 \\ {Factor}_{Genetic} \times {Factor}_{Citation} , 0 < {Factor}_{Genetic} \times {Factor}_{Citation} \leq 100\end{matrix} ,$$
where Scoreᵣᵢₛₖ represents the risk score, Factor_{Genetic} represents the genetic factor, and Factor_{citation} represents the citation factor.

7. A system for evaluating a risk of a subject getting a specific disease, comprising:
a storage configured to store a reference database that is established in advance by collecting data from a medical literature database, an allele frequency database, and a plurality of databases that compiles data of genome-wide association study (GWAS), the reference database containing M number of original parameter sets that respectively correspond to M number of specific risk alleles respectively at M number of chromosomal positions where single-nucleotide polymorphisms (SNPs) related to the specific disease occur, M being a positive integer greater than one, each of the M number of original parameter sets including a plurality of statistics related to the corresponding one of the M number of specific risk alleles, a global risk allele frequency that is related to an allele frequency of the corresponding one of the M number of specific risk alleles in global population, a group-specific risk allele frequency that is related to an allele frequency of the corresponding one of the M number of specific risk alleles in a certain race group, a global reference allele frequency that is related to the global risk allele frequency, a number of citation times that literatures related to the corresponding one of the M number of specific risk alleles are cited, and a number of chromosomes in a homologous chromosome pair having the corresponding one of the M number of specific risk alleles;
a receiving module configured to receive an SNP profile derived from genome sequencing data of the subject; and
a processor electrically connected to said storage and said receiving module, and configured to implement a method that includes steps of
selecting, from the SNP profile derived from genome sequencing data of the subject, N number of target alleles that respectively match N number of specific risk alleles in the M number of specific risk alleles indicated in the reference database, N being a positive integer not greater than M,
selecting, from among the M number of original parameter sets, N number of target parameter sets that correspond respectively to the N number of specific risk alleles,
calculating, for each of the N number of target parameter sets, a race factor based on the global risk allele frequency and the group-specific risk allele frequency of the target parameter set,
calculating a genetic factor based on the statistics respectively of the N number of target parameter sets, the global reference allele frequencies respectively of the N number of target parameter sets, the race factors respectively calculated for the N number of target parameter sets, and the numbers of chromosomes in homologous chromosome pairs of the N number of target parameter sets,
calculating a citation factor based on the numbers of citation times respectively of the N number of target parameter sets, and
calculating a risk score based on the genetic factor and the citation factor.

8. The system as claimed in claim 7, wherein for each of the M number of original parameter sets, the statistics include:
a p-value that represents a probability of an association between the specific disease and the corresponding one of the M number of specific risk alleles is due to random chance; and
an odds ratio, which is a ratio of a probability of a person with the corresponding one of the M number of specific risk alleles getting the specific disease to a probability of a person without the corresponding one of the M number of specific risk alleles getting the specific disease.

9. The system as claimed in claim 8, wherein the step of calculating a genetic factor is to calculate the genetic factor according to a formula:
$${Factor}_{Genetic} = \frac{1}{M} {Σ}_{i = 1}^{N} \frac{- {log}_{10} {P}_{i} \times {OR}_{i} \times {SNP\_Type}_{i} \times {Factor}_{R ace , i}}{{Frequency}_{G l obal ref , i}} ,$$
where Factor_{Genetic} represents the genetic factor, Pᵢ represents the p-value for an i^{th} one of the N number of specific risk alleles, ORᵢ represents the odds ratio for the i^{th} one of the N number of specific risk alleles, SNP_Typeᵢ represents the number of chromosomes in a homologous chromosome pair having the i^{th} one of the N number of specific risk alleles, Factor_{Race,i} represents the race factor for the i^{th} one of the N number of specific risk alleles, and Frequency_{Global ref,i} represents the global reference allele frequency for the i^{th} one of the N number of specific risk alleles.

10. The system as claimed in claim 7, wherein for an i^{th} one of the target parameter sets that corresponds to an i^{th} one of the N number of specific risk alleles, i being an integer ranging from one to N, the step of calculating a race factor is to calculate the race factor according to formulas:
$${Factor}_{R a c e , i} = \{ \begin{matrix}{log}_{10} {Frequency\_ratio}_{Group risk , i} + 1 , {log}_{10} {Frequency\_ratio}_{Group risk , i} \geq 0 \\ \frac{1}{1 - {log}_{10} {Frequency\_ratio}_{Group risk , i}} , {log}_{10} {Frequency\_ratio}_{Group risk , i} < 0\end{matrix} ;$$
$$and {Frequency\_ratio}_{Group risk , i} = \frac{{Frequency}_{Group risk , i}}{{Frequency}_{G l obal risk , i}} ,$$
where Factor_{Race,i} represents the race factor for the i^{th} one of the N number of specific risk alleles, Frequency_{Group risk,i} represents the group-specific risk allele frequency for the i^{th} one of the N number of specific risk alleles, and Frequency_{Global risk,i} represents the global risk allele frequency for the i^{th} one of the N number of specific risk alleles.

11. The system as claimed in claim 7, wherein the step of calculating a citation factor is to calculate the citation factor according to a formula:
Factor_{citation}=lnΣᵢ₌₁^{N}(Citation_numᵢ+1),
where Factor_{citation} represents the citation factor, and Citation_numᵢ represents the number of citation times for an i^{th} one of the N number of specific risk alleles.

12. The system as claimed in claim 7, wherein the step of calculating a risk score is to calculate the risk score based on a formula:
$${Score}_{risk} = \{ \begin{matrix}100 , {Factor}_{Genetic} \times {Factor}_{Citation} > 100 \\ {Factor}_{Genetic} \times {Factor}_{Citation} , 0 < {Factor}_{Genetic} \times {Factor}_{Citation} \leq 100\end{matrix} ,$$
where Scoreᵣᵢₛₖ represents the risk score, Factor_{Genetic} represents the genetic factor, and Factor_{citation} represents the citation factor.
